(19) United States
(12) Patent Application Publication
(10) Pub. No.: US 2023/0333117 A1
(43) Pub. Date: Oct. 19, 2023

(54) METHODS OF DETERMINING RISK OF SUICIDE BY IMMUNOLOGICAL ASSESSMENT

(71) Applicant: THERAPEUTIC SOLUTIONS INTERNATIONAL, INC., Oceanside, CA (US)

(72) Inventors: Thomas E. ICHIM, Oceanside, CA (US); Timothy G. DIXON, Oceansdie, CA (US); Kalina O'CONNOR, Oceansdie, CA (US); Famela RAMOS, Oceansdie, CA (US); James VELTMEYER, Oceansdie, CA (US); Wais KAIHANI, Oceansdie, CA (US)

(73) Assignee: THERAPEUTIC SOLUTIONS INTERNATIONAL, INC., Oceanside, CA (US)

(21) Appl. No.: 18/042,437

(22) PCT Filed: Aug. 23, 2021
(86) PCT No.: PCT/US2021/047209

§ 371 Date: Feb. 21, 2023

Related U.S. Application Data
(60) Provisional application No. 63/068,388, filed on Aug. 21, 2020.

(51) Int. Cl. G01N 33/68

(52) U.S. Cl. CPC G01N 33/6869; G01N 2800/30; G01N 2800/50

(57) ABSTRACT

Disclosed are means and methods of identifying risk of suicide and/or suicidal ideation by assessment of immunologically related cytokines and cells. In one embodiment, a score, termed the “Campbell Score” is devised based on assessment of serum cytokines, ability of immune cells to make cytokines when stimulated ex vivo, and ability of immune cells to produce neurotransmitters when stimulated ex-vivo. In on embodiment the concentration of interleukin-6 is utilized as a means of assessing suicidal propensity along, and/or in combination with metabolites of the enzyme indolamine 2,3 deoxygenase.

### CROSS-REFERENCE TO RELATED APPLICATIONS

The present application claims the benefit of U.S. Provisional Application No. 63/068,388, filed Aug. 21, 2020, the contents of which are incorporated herein by reference.

### BACKGROUND

Suicide is defined as the intentional termination of one's own life and constitutes the 14th leading cause of global years of life lost. According to a recent report by the World Health Organization, over 800 000 deaths by suicide occur around the world each year, but the actual number may be higher. Cultural taboos and the fact that suicide is considered a criminal act in some countries may affect the statistical reporting of such events. Suicide attempts are estimated to be 10 to 20 times more frequent than the number of completed suicides. Both deaths by suicide and attempts are signs of severe psychological suffering, and a completed suicide carries with it an emotional burden that can impact family for years to come.

The pharmacological treatment of depressed and suicidal individuals has increased over the past decades, but the incidence rates of suicide and suicide attempts are still increasing. A total of 1.5 million people will die from suicide in 2020, if the current trends remain unaltered. Accurate suicide risk determination is a very difficult task for clinicians, considering that a patient at high risk for suicide is likely to minimize this symptom. The healthcare system is in many cases unable to accurately detect suicidality, in spite of the fact that almost half of the suicidal patients actually contact healthcare providers in the months prior to their attempt. Consequently, there is a great need for both improved methods for the detection of suicide risk and for effective, novel pharmacological treatment options for suicidal patients.

The pharmacological treatment options for suicidal patients today are likely to depend on the patient's primary psychiatric diagnosis and often include antidepressants and anxiolytic medications. The positive effects of pharmacotherapy on symptoms of depression take weeks to months to develop, and moreover, there may be an increased risk for suicidal behavior during this initial period of treatment, especially in children, adolescents and adults up to age 25. Besides the conventional antidepressant drugs, clozapine (in schizophrenia) and lithium (in patients with mood disorders) are sometimes indicated specifically for reduction of suicide risk. So-called treatment-resistant depression, which often includes symptoms of suicidality, can be treated with electroconvulsive therapy in many countries, in addition to pharmacological treatment. Accumulating studies suggest that subanesthetic doses of intravenous ketamine exerts rapid antidepressant and anti-suicidal effects. The underlying biological mechanisms by which these treatment options modulate suicidal symptoms are not fully understood.

Unfortunately, there are no clinically useful objective means of determining predisposition to suicide or suicidal ideations.

### DESCRIPTION

The invention provides assessment of inflammatory cytokines from system and/or local sources as a means of quantifying predisposition to suicide and/or suicidal ideation. In one embodiment the invention provides for ex vivo activation of lymphocytes and assessment of inflammatory cytokine production from said lymphocytes. In patients predisposed to producing higher levels of inflammatory cytokines, there is an increased risk of suicide or suicidal ideation.

In one embodiment the invention provides methods for using biomarkers and a multi-step algorithm to determine a diagnosis of enhanced predisposition to suicide. The methods can include, for example, selecting a panel of biomarkers related to suicide, obtaining clinical data from subjects for the biomarkers, and applying an optimization algorithm to the clinical data in order to arrive at coefficients for the panel of selected biomarkers. As described herein, the panel was created using the biomarker measurements and coefficients for individuals known to have suicide and those who do not have the condition. In some embodiments of this methodology, for example, algorithms incorporating data from multiple biomarkers biological samples such as serum or plasma can be developed for patient stratification, identification of pharmacodynamic markers, and monitoring treatment outcome.

In one aspect, this document features a method for assessing the likelihood that an individual has suicide. The method can include (a) identifying groups of biomarkers that may be related to suicide; (b) measuring the level of each of the biomarkers biological samples from a plurality of subjects, wherein some of the subjects are diagnosed as having suicide and some of the subjects do not have suicide; (c) applying a normalization function to the measured level of each of the biomarkers; (d) applying an optimization algorithm to the measured biomarker levels and calculating coefficients for selected biomarkers within each group; (e) calculating the result of the algorithm for the individual to determine whether the individual is likely to have suicide or is not likely to have suicide. The groups of biomarkers can include two or more inflammatory biomarkers, HPA axis biomarkers, metabolic biomarkers, or neurotrophic biomarkers. The inflammatory biomarkers can be selected from the group consisting of alpha 1 antitrypsin, alpha 2 macroglobulin, CD40 ligand, interleukin 6, interleukin 13, interleukin 18, interleukin 1 receptor antagonist, myeloperoxidase, plasminogen activator RANTES, and tumor necrosis factor alpha (TNF-.alpha.), and soluble TNF-.alpha. receptor type II. The HPA axis biomarkers can be selected from the group consisting of cortisol, epidermal growth factor, granulocyte colony stimulating factor, pancreatic polypeptide, adrenocorticotropic hormone, arginine vasopressin, and corticotropin-releasing hormone. The metabolic biomarkers can be selected from the group consisting of adiponectin, acylation stimulating protein, apolipoprotein CIII, fatty acid binding protein, insulin, leptin, prolactin, resistin, testosterone, and thyroid stimulating hormone. The neurotrophic biomarkers can be selected from the group consisting of brain-derived neurotrophic factor, S100B, neurotrophin 3, glial cell line-derived neurotrophic factor, and artemin. The group of biomarkers can consist of alpha-1 antitrypsin, apolipoprotein CIII, brain derived neurotrophic factor, cortisol, epidermal growth factor, myeloperoxidase, prolactin, resistin, and soluble tumor necrosis factor receptor type II.

In one embodiment assessment of interleukin-6 is provided as a means of quantifying suicidal risk. IL-6 may be measured in blood, plasma, urine, cerebrospinal fluid, and saliva. Quantification of IL-6 as well as other inflammatory cytokines may be performed using a variety of means known in the art. In one embodiment, quantification of cytokines is performed by enzyme linked immunosorbent assay (ELISA).

In one embodiment, inflammatory cytokines are used to detect “major depressive disorder”, or MDD, is characterized as a psychiatric disorder meeting five criteria: 1) the presence during the same 2 week period which together represent a change from previous functioning, of a depressed/sad mood or a loss of interest and pleasure, together with five (or more) of the following additional criteria occurring nearly every day i) depressed/sad mood ii) loss of interest and pleasure iii) significant weight loss when not dieting or weight gain or a decrease or increase in appetite iv) insomnia or hypersomnia v) psychomotor agitation or retardation vi) fatigue or loss of energy vii) feelings of worthlessness or excessive or inappropriate guilt

viii) diminished ability to think or concentrate or indecisiveness ix) recurrent thoughts of death or suicidal ideation, planning or attempt: 2) the symptoms cause clinically significant distress or impairment in social, occupational or other functioning: 3) the episode is not better accounted for by a psychotic disorder: 4) the episode is not attributable to the physiological effects of a substance or to another medical condition: 5) there has never been a manic or hypomanic episode (Diagnostic and Statistical Manual of Mental Disorders, 5th Edition, American Psychiatric Association, 2013). Other indications contemplated include treating, preventing, or ameliorating one or more symptoms of a disorder including, but not limited to, Rett syndrome, depression, refractory depression, suicidality, obsessive-compulsive disorder, fibromyalgia, post-traumatic stress syndrome, autism spectrum disorder, and depression associated with genetic disorders.

In one embodiment, the major depressive disorder is with anxious distress. In another embodiment, the disorder is with mixed features. In another embodiment, the disorder is with melancholic features. In another embodiment, the disorder is with atypical features. In another embodiment, the disorder is with mood-congruent psychotic features. In another embodiment, the disorder is with mood-incongruent psychotic features. In another embodiment, the disorder is with catatonia. In another embodiment, the disorder is with peripartum onset. In another embodiment, the disorder is with seasonal pattern.

Major depressive disorder (MDD) is characterized by persistent lack of interest in physical and mental activities, and causes detrimental changes in normal life, thereby creating a burden on the family and society. Clinical studies have shown that classical anti-depressant drugs that reduce depression are effective in relatively less percentage of MDD patients. These drugs also take days to weeks to elicit the antidepressant effect. Remarkably, however, a single intravenous administration of ketamine, an antagonist of ionotropic glutamatergic N-methyl-D-aspartate receptor (NMDAR), is sufficient to reduce depression in human patients and its effect also persists for many hours to days.

Investigations trying to understand the cellular and molecular mechanisms of ketamine action has brought several key insights: ketamine administration leads to rapid synthesis of proteins such as brain-derived neurotrophic factor (BDNF) and GluA1 subunit of .alpha.-amino-.beta.-hydroxy-5-methyl-4-isoxazolepropionic acid receptor (AMPAR). Up-regulated BDNF together with other dendritic/synaptic proteins such as GluA1 trigger evoked synaptic transmission to mediate the long-lasting effects of ketamine. Ketamine-mediated new synthesis of BDNF requires the deactivation of eEF2K (also known as calcium/calmodulin dependent kinase III; a translation regulation kinase involved in the elongation phase), and subsequently reduced phosphorylation of eEF2 on Thr-56. This mechanistic insight is further supported by the findings that eEF2K inhibitors induce fast behavioral antidepressant effects in mice.

In other aspects, the benefits achieved using the methods of the disclosure with outweigh any adverse events in 12 lead ECG findings, method discontinuation, Digit Symbol Substitution Test (DSST), reaction time test (Cambridge COGNITION and/or Cogstate battery), self-administered Stanford sleepiness scale, a Bladder Pain/Interstitial Cystitis Symptom Score (BPIC-SS), a Modified Observer's Alertness/Sedation Scale (MOAA/S), a Clinician-Administered Dissociative States Scale (CADS S), a Suicidality Scale-Clinician-Rated Columbia Suicide Severity Rating Scale (C-SSRS), 4 items positive symptoms subscale from the Brief Psychiatric Rating Scale (BPRS), and/or 20 item Physician Withdrawal Checklist (PWC-20), as compared to placebo. In other aspects, the benefits achieved using the methods of the disclosure with outweigh any adverse events in 12 lead ECG findings, method discontinuation, Digit Symbol Substitution Test (DSST), reaction time test (Cambridge COGNITION and/or Cogstate battery), self administered Stanford sleepiness scale, a Bladder Pain/Interstitial Cystitis Symptom Score (BPIC-SS), a Modified Observer's Alertness/Sedation Scale (MOAA/S), a Clinician-Administered Dissociative States Scale (CADSS), a Suicidality Scale-Clinician-Rated Columbia Suicide Severity Rating Scale (C-SSRS), 4 items positive symptoms subscale from the Brief Psychiatric Rating Scale (BPRS), and/or 20 item Physician Withdrawal Checklist (PWC-20), as compared to other methods of treating MDD, including treatment-resistant MDD.

In one embodiment of the invention, the quantification of inflammation is utilized to guide treatment for stimulation of neurogenesis. Specifically, the study of adult neurogenesis has been previously described [1-4], and suppression of neurogenesis in alcohol, opioid, cocaine and methamphetamine addiction has been previously reported [5-15]. By quantifying and reducing the inflammation in the body and specifically in the brain, restoration of neurogenesis is envisioned. In one embodiment of the invention, stem cells are utilized to overcome drug induced neurotoxicity [16], and said probiotic/enzyme mixture is utilized to enhance efficacy of stem cells. Enhancement of efficacy comprises stimulation of growth factor production, inhibition of inflammation and triggering of mitogenesis of stem cells and existing endogenous regenerative cells.

In another embodiment, the quantification of inflammation is utilized to guide treatment with suicide preventative and/or drug addiction preventative medications such as growth hormone [17], agents which block adrenal hormones [18, 19], enhancement of serotonin [20-22], administration of FGF-2 [23, 24], antidepressants such as tranylcypromine, reboxetine, fluoxetine, haloperidol, tranylcypromine, reboxetine, fluoxetine, and haloperidol [25], electroconvulsive therapy [26], lithium [27], insulin like growth factor [28], inhibition of IL-6 [29], heparin binding epidermal growth factor like growth factor [30], VEGF [31], DHEA [32], BDNF [33], NMDA receptor antagonists [34], PGE2 [35], prolactin [36], the Chronic AMPA receptor potentiator (LY451646) [37], PACAP [38], lithium [39], transcranial magnetic field stimulation [40], olanzapine or fluoxetine [41].

### Example 1

Clinical Trial Association Between Plasma Interleukin-6 and Suicidal Ideation

A clinical trial was conducted, which was registered on the Federal Clinical Trials website (#NCT04606875), and assessed levels of inflammatory marker interleukin-6 in 10 patients with no history of suicide (Group 1), 10 patients with suicidal ideations (Group 2), and 10 patients with suicidal ideations who attempted suicide in the last 6 months (Group 3). Interleukin-6 was measured from plasma that was treated with n-acetylcysteine at a concentration of 10 ng/ml. Assessment of concentration was performed by ELISA. Patients for the suicidal ideation group (Group 2) were selected using the (Hamilton Depression Rating Scale suicide item >2) and reported low acquired capability for suicide (Acquired Capability for Suicide Scale <20). Patients in the suicidal ideations with attempted suicide group (Group 3) were selected using (Hamilton Depression Rating Scale suicide item >2) and reported high acquired capability for suicide (Acquired Capability for Suicide Scale >60)

Results demonstrated that patients in Group 1 had 7.6±2.4 pg/ml of cytokine, whereas Group 2 had 28.9±6.3 pg/ml and in Group 3 had 45.8±7.7 pg/ml.

### REFERENCES

1. Kaplan M S, Hinds J W: Neurogenesis in the adult rat: electron microscopic analysis of lightradioautographs. Science 1977, 197(4308):1092-1094.
2. Lubbers K, Wolff J R, Frotscher M: Neurogenesis of GABAergic neurons in the rat dentategyrus: a combined autoradiographic and immunocytochemical study. Neurosci Lett 1985, 62(3):317-322.
3. Eriksson P S, Perfilieva E, Bjork-Eriksson T, Alborn A M, Nordborg C, Peterson D A, Gage F H: Neurogenesis in the adult human hippocampus. Nat Med 1998, 4(11):1313-1317.
4. Roy N S, Wang S, Jiang L, Kang J, Benraiss A, Harrison-Restelli C, Fraser R A, Couldwell W T, Kawaguchi A, Okano H et al: In vitro neurogenesis by progenitor cells isolated from the adult human hippocampus. Nat Med 2000, 6(3):271-277.
5. Hauser K F, Houdi A A, Turbek C S, Elde R P, Maxson W, 3rd: Opioids intrinsically inhibit the genesis of mouse cerebellar granule neuron precursors in vitro: differential impact of mu and delta receptor activation on proliferation and neurite elongation. Eur J Neurosci 2000, 12(4):1281-1293.
6. Eisch A J, Mandyam C D: Drug dependence and addiction, II: Adult neurogenesis and drug abuse. Am J Psychiatry 2004, 161(3):426.
7. Fischer S J, Arguello A A, Charlton J J, Fuller D C, Zachariou V, Eisch A J: Morphine blood levels, dependence, and regulation of hippocampal subgranular zone proliferation rely on administration paradigm. Neuroscience 2008, 151(4):1217-1224.
8. Cunha-Oliveira T, Rego A C, Oliveira C R: Cellular and molecular mechanisms involved in the neurotoxicity of opioid and psychostimulant drugs. Brain Res Rev 2008, 58(1):192-208.
9. Jin J, Kittanakom S, Wong V, Reyes B A, Van Bockstaele E J, Stagljar I, Berrettini W, Levenson R: Interaction of the mu-opioid receptor with GPR177 (Wntless) inhibits Wnt secretion: potential implications for opioid dependence. BMC Neurosci 2010, 11:33.
10. Zheng H, Law P Y, Loh H H: Non-Coding RNAs Regulating Morphine Function: With Emphasis on the In vivo and In vitro Functions of miR-190. Front Genet 2012, 3:113.
11. Hafizi M, Bakhshandeh B, Soleimani M, Atashi A: Exploring the enkephalinergic differentiation potential in adult stem cells for cell therapy and drug screening implications. In Vitro Cell Dev Biol Anim 2012, 48(9):562-569.
12. Zheng H, Zhang Y, Li W, Loh H H, Law P Y: NeuroD modulates opioid agonist-selective regulation of adult neurogenesis and contextual memory extinction. Neuropsychopharmacology 2013, 38(5):770-777.
13. Bernstein H G, Trubner K, Krebs P, Dobrowolny H, Bielau H, Steiner J, Bogerts B: Increased densities of nitric oxide synthase expressing neurons in the temporal cortex and the hypothalamic paraventricular nucleus of polytoxicomanic heroin overdose victims: possible implications for heroin neurotoxicity. Acta Histochem 2014, 116(1):182-190.
14. Teuchert-Noodt G, Dawirs R R, Hildebrandt K: Adult treatment with methamphetamine transiently decreases dentate granule cell proliferation in the gerbil hippocampus. J Neural Transm (Vienna) 2000, 107(2):133-143.
15. Yamaguchi M, Suzuki T, Seki T, Namba T, Juan R, Arai H, Hori T, Asada T: Repetitive cocaine administration decreases neurogenesis in adult rat hippocampus. Ann N Y Acad Sci 2004, 1025:351-362.
16. Kazma M, Izrael M, Revel M, Chebath J, Yanai J: Survival, differentiation, and reversal of heroin neurobehavioral teratogenicity in mice by transplanted neural stem cells derived from embryonic stem cells. J Neurosci Res 2010, 88(2):315-323.
17. Rhodin A, von Ehren M, Skottheim B, Gronbladh A, Ortiz-Nieto F, Raininko R, Gordh T, Nyberg F: Recombinant human growth hormone improves cognitive capacity in a pain patient exposed to chronic opioids. Acta Anaesthesiol Scand 2014, 58(6):759-765.
18. Cameron H A, Gould E: Adult neurogenesis is regulated by adrenal steroids in the dentategyrus. Neuroscience 1994, 61(2):203-209.
19. Montaron M F, Petry K G, Rodriguez J J, Marinelli M, Aurousseau C, Rougon G, Le Moal M, Abrous D N: Adrenalectomy increases neurogenesis but not PSA-NCAM expression in ageddentate gyrus. Eur J Neurosci 1999, 11(4):1479-1485.
20. Brezun J M, Daszuta A: Depletion in serotonin decreases neurogenesis in the dentate gyrus and the subventricular zone of adult rats. Neuroscience 1999, 89(4):999-1002.
21. Gould E: Serotonin and hippocampal neurogenesis. Neuropsychopharmacology 1999, 21(2Supp1):46S-51S.
22. Brezun J M, Daszuta A: Serotonin may stimulate granule cell proliferation in the adult hippocampus, as observed in rats grafted with foetal raphe neurons. Eur J Neurosci 2000, 12(1):391-396.
23. Wagner J P, Black I B, DiCicco-Bloom E: Stimulation of neonatal and adult brain neurogenesis by subcutaneous injection of basic fibroblast growth factor. J Neurosci 1999, 19(14):6006-6016.
24. Palmer T D, Markakis E A, Willhoite A R, Safar F, Gage F H: Fibroblast growth factor-2 activates a latent neurogenic program in neural stem cells from diverse regions of the adult CNS. J Neurosci 1999, 19(19):8487-8497.
25. Malberg J E, Eisch A J, Nestler E J, Duman R S: Chronic antidepressant treatment increases neurogenesis in adult rat hippocampus. J Neurosci 2000, 20(24):9104-9110.
26. Madsen T M, Treschow A, Bengzon J, Bolwig T G, Lindvall O, Tingstrom A: Increased neurogenesis in a model of electroconvulsive therapy. Biol Psychiatry 2000, 47(12):1043-1049.
27. Chen G, Rajkowska G, Du F, Seraji-Bozorgzad N, Manji H K: Enhancement of hippocampal neurogenesis by lithium. J Neurochem 2000, 75(4):1729-1734.
28. O'Kusky J R, Ye P, D'Ercole A J: Insulin-like growth factor-I promotes neurogenesis and synaptogenesis in the hippocampal dentate gyrus during postnatal development. J Neurosci 2000, 20(22):8435-8442.
29. Vallieres L, Campbell I L, Gage F H, Sawchenko P E: Reduced hippocampal neurogenesis in adult transgenic mice with chronic astrocytic production of interleukin-6. J Neurosci 2002, 22(2):486-492.
30. Jin K, Mao XO, Sun Y, Xie L, Jin L, Nishi E, Klagsbrun M, Greenberg D A: Heparin-binding epidermal growth factor-like growth factor: hypoxia-inducible expression in vitro and stimulation of neurogenesis in vitro and in vivo. J Neurosci 2002, 22(13):5365-5373.
31. Jin K, Zhu Y, Sun Y, Mao XO, Xie L, Greenberg D A: Vascular endothelial growth factor (VEGF) stimulates neurogenesis in vitro and in vivo. Proc Natl Acad Sci USA 2002, 99(18):11946-11950.
32. Karishma K K, Herbert J: Dehydroepiandrosterone (DHEA) stimulates neurogenesis in the hippocampus of the rat, promotes survival of newly formed neurons and prevents corticosterone-induced suppression. Eur J Neurosci 2002, 16(3):445-453.
33. Lee J, Duan W, Mattson M P: Evidence that brain-derived neurotrophic factor is required for basal neurogenesis and mediates, in part, the enhancement of neurogenesis by dietary restriction in the hippocampus of adult mice. J Neurochem 2002, 82(6):1367-1375.
34. Nacher J, Alonso-Llosa G, Rosell D R, McEwen B S: NMDA receptor antagonist treatment increases the production of new neurons in the aged rat hippocampus. Neurobiol Aging 2003, 24(2):273-284.
35. Uchida K, Kumihashi K, Kurosawa S, Kobayashi T, Itoi K, Machida T: Stimulatory effects of prostaglandin E2 on neurogenesis in the dentate gyrus of the adult rat. Zoolog Sci 2002, 19(11):1211-1216.
36. Shingo T, Gregg C, Enwere E, Fujikawa H, Hassam R, Geary C, Cross J C, Weiss S: Pregnancy-stimulated neurogenesis in the adult female forebrain mediated by prolactin. Science 2003, 299(5603):117-120.
37. Bai F, Bergeron M, Nelson D L: Chronic AMPA receptor potentiator (LY451646) treatment increases cell proliferation in adult rat hippocampus. Neuropharmacology 2003, 44(8):1013-1021.
38. Mercer A, Ronnholm H, Holmberg J, Lundh H, Heidrich J, Zachrisson O, Ossoinak A, Frisen J, Patrone C: PACAP promotes neural stem cell proliferation in adult mouse brain. J NeurosciRes 2004, 76(2):205-215.
39. Kim J S, Chang M Y, Yu I T, Kim J H, Lee S H, Lee Y S, Son H: Lithium selectively increases neuronal differentiation of hippocampal neural progenitor cells both in vitro and in vivo. J Neurochem 2004, 89(2):324-336.
40. Arias-Carrion O, Verdugo-Diaz L, Feria-Velasco A, Millan-Aldaco D, Gutierrez A A, Hernandez-Cruz A, Drucker-Colin R: Neurogenesis in the subventricular zone following transcranial magnetic field stimulation and nigrostriatal lesions. J Neurosci Res 2004, 78(1):16-28.
41. Kodama M, Fujioka T, Duman R S: Chronic olanzapine or fluoxetine administration increases cell proliferation in hippocampus and prefrontal cortex of adult rat. Biol Psychiatry 2004, 56(8):570-580.

1. A method of predicting propensity for suicide and/or suicidal ideation comprising the steps of: a) selecting a patient at potential risk of suicide/suicidal ideation; b) obtaining a biological fluid from said patient; c) analyzing immunological molecules and/or immunological cells from said patient; and d) making a prediction of suicidal risk/suicidal ideation based on immunological molecules/immunological cells.

2. The method of claim 1, wherein said patient at potential risk of suicide is a healthy individual.

3. The method of claim 1, wherein said patient at potential risk of suicide is a victim of major depressive disorder.

4. The method of claim 1, wherein said patient at potential risk of suicide suffers from chemical addictions.

5. The method of claim 4, wherein said chemical addictions are selected from a group of additions comprising of: a) cocaine addiction; b) alcohol addiction; c) methamphetamine addiction; d) cannabis addiction; e) nicotine addiction and f) psychedelic mushroom addiction.

6. The method of claim 1, wherein said biological fluid is urine.

7. The method of claim 1, wherein said biological fluid is blood.

8. The method of claim 1, wherein said biological fluid is cerebral spinal fluid.

9. The method of claim 1, wherein said biological fluid is saliva

10. The method of claim 1, wherein said immunological molecule is a cytokine.

11. The method of claim 1, wherein a serum concentration of more than 10% higher interleukin 1 beta in a patient compared to an age-matched healthy control is considered to be associated with an increased risk of suicide and/or suicidal ideation.

12. The method of claim 1, wherein a serum concentration of more than 10% higher interleukin-6 in a patient compared to an age-matched healthy control is considered to be associated with an increased risk of suicide and/or suicidal ideation.

13. The method of claim 1, wherein a serum concentration of more than 10% higher interleukin-8 in a patient compared to an age-matched healthy control is considered to be associated with an increased risk of suicide and/or suicidal ideation.

14. The method of claim 1, wherein a serum concentration of more than 10% higher interleukin-11 in a patient compared to an age-matched healthy control is considered to be associated with an increased risk of suicide and/or suicidal ideation.

15. The method of claim 1, wherein a serum concentration of more than 10% higher interleukin-12 in a patient compared to an age-matched healthy control is considered to be associated with an increased risk of suicide and/or suicidal ideation.

16. The method of claim 1, wherein a serum concentration of more than 10% higher interleukin-15 in a patient compared to an age-matched healthy control is considered to be associated with an increased risk of suicide and/or suicidal ideation.

17. The method of claim 1, wherein a serum concentration of more than 10% higher interleukin-9 in a patient compared to an age-matched healthy control is considered to be associated with an increased risk of suicide and/or suicidal ideation.

18. The method of claim 1, wherein a serum concentration of more than 10% higher interleukin-17 in a patient compared to an age-matched healthy control is considered to be associated with an increased risk of suicide and/or suicidal ideation.

19. The method of claim 1, wherein a serum concentration of more than 10% higher interleukin-18 in a patient compared to an age-matched healthy control is considered to be associated with an increased risk of suicide and/or suicidal ideation.

20. The method of claim 1, wherein a serum concentration of more than 10% higher interleukin-23 in a patient compared to an age-matched healthy control is considered to be associated with an increased risk of suicide and/or suicidal ideation.

21. (canceled)

22. (canceled)

23. (canceled)

24. (canceled)

25. (canceled)

26. (canceled)

27. (canceled)
